(19) United States
(12) Patent Application Publication
(10) Pub. No.: US 2022/0047496 A1
(43) Pub. Date: Feb. 17, 2022

(54) COSMETIC COMPOSITION FOR SKIN CARE

(71) Applicant: L'OREAL, Paris (FR)

(72) Inventors: Yirui ZHU, Shanghai (CN); Ruijun CHEN, Shanghai (CN); Yangdong Mellon CHEN, Shanghai (CN)

(73) Assignee: L'OREAL, Paris (FR)

(21) Appl. No.: 17/277,124

(22) PCT Filed: Oct. 30, 2018
(86) PCT No.: PCT/CN2018/112667

§ 371 Date: Mar. 17, 2021

(51) Int. Cl. A61K 8/99; A61K 8/06; A61K 8/9794; A61K 8/9789; A61K 8/365; A61K 8/60; A61Q 19/00

(52) U.S. Cl. CPC A61K 8/99; A61K 8/062; A61K 8/9794; A61Q 19/00; A61K 8/365; A61K 8/60; A61K 8/9789

(57) ABSTRACT

Disclosed herein are a cosmetic composition in form of oil-in-water emulsion comprises: (i) at least one structuring agent with a melting point of greater than or equal to 50° C., (ii) at least one structuring agent with a melting point of less than or equal to 45° C., (iii) at least one amino acid surfactant; (iv) at least one non-ionic surfactant of ester type, comprising a mixture of at least one monounsaturated ester and at least one polyglyceryl diester; and (v) at least one acidic cosmetic active ingredient and a method for caring for the skin, comprising the application to the skin of said cosmetic composition

### TECHNICAL FIELD

The present invention relates to a cosmetic composition. In particular, the present invention relates to a cosmetic composition for caring for keratin materials, especially skin.

### BACKGROUND ART

The development of formulations dedicated to caring for and/or making up the skin and/or lips, is permanent. Said formulations have to show satisfactory properties in terms of application, such as easy usage, and also in terms of sensory, such as hydration and/or moisturizing feeling after application.

The skin is the protective barrier for the human body. It protects the interior of the body from physical injury (such as trauma) and biological injury (such as bacteria, viruses or fungi). The skin of the human body comprises the dermis and mthe epidermis. The epidermis is the top most layer of the skin, and its superficial layer is called the stratum corneum.

Moisturization is provided to the skin by the water in the deep-lying layers and by perspiration. Cutaneous moisturization disorders, and especially skin dryness, are often observed with age and/or changes in climate. However, such conditions may also be manifested in young individuals.

A wide variety of cosmetic compositions have been used to care for the skin, in particular to provide moisturizing or hydration to the skin. These compositions typically contain lipophilic moisturizing agents that inhibit water loss via occlusion. These compositions can additionally comprise other skin beneficial agents such as vitamins and humectants. Addition of these ingredients can increase the moisturization of the skin. For example, efforts have been made to formulate compositions in the form of creams or emulsions. Thanks to the thick texture of the compositions, it is possible to provide to the skin moisturizing feeling via occlusion. The emulsions are usually in the form of water-in-oil emulsion.

However, these types of products are still not satisfying.

Consumers feel an unappealing greasy after application of the product. Moreover, due to the thick texture of the products, it is not easy to apply on the skin evenly.

In addition, some acidic active ingredients are known for their anti-aging effect. However, the addition of such acidic active ingredients will adversely impact the stability of the composition.

There is thus still a need to formulate a stable composition for caring for the skin, which has good cosmetic properties, in particular anti-aging, moisturizing and/or hydration of the skin without greasy feeling.

Besides, the composition is expected to be easily spread on the skin during application.

It is therefore desirable to provide skin care compositions that present excellent application and sensory characteristics as well as anti-aging effect.

### SUMMARY OF THE INVENTION

The Applicant has now discovered that it is possible to formulate such compositions having the desired properties as described above.

Specifically, the Applicant has discovered that it is possible to formulate stable compositions for caring for and/or making up of keratin materials, which confer a fresh feeling and anti-aging effect after application onto skin.

Accordingly, the present invention relates to a cosmetic composition in form of an oil-in-water emulsion, comprising:

(i) at least one structuring agent with a melting point of greater than or equal to 50° C.,

(ii) at least one structuring agent with a melting point of less than or equal to 45° C.,

(iii) at least one amino acid surfactant;

(iv) at least one non-ionic surfactant of ester type, comprising a mixture of at least one monounsaturated ester and at least one polyglyceryl diester; and

(v) at least one acidic cosmetic active ingredient.

The cosmetic composition of the present invention is in form of an oil-in-water emulsion. Thus, said cosmetic composition comprises a continuous aqueous phase and a dispersed fatty phase.

The composition of the present invention is easy to apply evenly on the skin, without long term massaging by fingers.

The cosmetic composition of the present invention presents an improved usage or application and sensory as compared to skin care compositions currently on the market, it is very easy to spread, while bringing a fresh and moisturizing feeling and anti-aging effect after application.

The composition according to the present invention as described above is stable over time at room temperature (25° C.), for example, after storage for 2 months, and further, for example, for 4 months.

### BRIEF DESCRIPTION OF THE FIRGURES

Other subjects and characteristics, aspects and advantages of the present invention will emerge even more clearly on reading the detailed description, the examples as well as the drawings that follow, wherein:

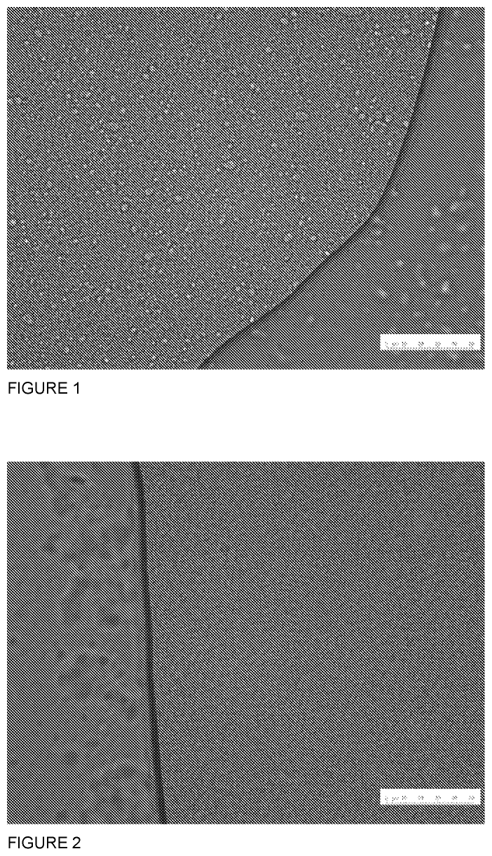

FIG. 1 is a micrograph of the cosmetic composition according to the comparative formula prepared in Example 1.

FIG. 2 is a micrograph of the cosmetic composition according to the inventive formula 1 prepared in Example 1.

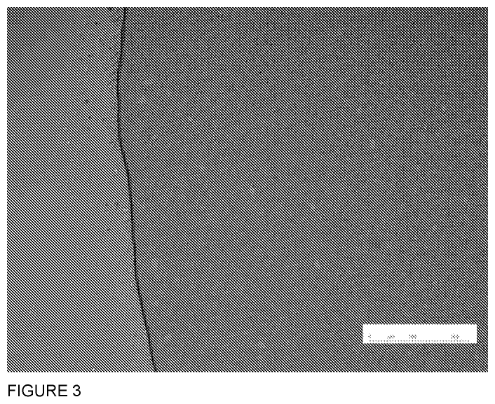

FIG. 3 is a micrograph of the cosmetic composition according to the inventive formula 2 prepared in Example 1.

### DETAILED DESCRIPTION OF THE INVENTION

In that which follows and unless otherwise indicated, the limits of a range of values are included within this range, in particular in the expressions “of between” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

### Aqueous Phase

The cosmetic composition of the present invention comprises a continuous aqueous phase.

Said aqueous phase is preferably present in an amount ranging from 10% to 99% by weight, more preferably from 20% to 90% by weight, and even more preferably from 50% to 85% by weight of the total weight of the composition.

Water is preferably present in the composition of the present invention in an amount ranging from 1% to 80% by weight, preferably from 5% to 77% by weight, more preferably from 10% to 75% by weight, relative to the total weight of the composition.

The continuous aqueous phase may comprise water, at least one organic solvent miscible with water or mixtures thereof.

Preferably, the continuous hydrophilic phase comprises at least one organic solvent miscible with water (at room temperature 25° C.) such as for example monoalcohols having from 2 to 6 carbon atoms such as ethanol, isopropanol; polyols having from 2 to 20 carbon atoms, preferably from 2 to 10 carbon atoms, and preferentially having from 2 to 6 carbon atoms, such as glycerol, propylene glycol, butylene glycol, pentylene glycol, hexylene glycol, caprylylglycol, dipropylene glycol, diethylene glycol; glycol ethers (notably having from 3 to 16 carbon atoms) such as mono-, di- or tri- propylene glycol (C₁-C₄)alkyl ethers, mono-, di- or tri-ethylene glycol (C₁-C₄) alkyl ethers and mixtures thereof.

The continuous hydrophilic phase of the composition of the present invention may comprise water and a polyol, preferably glycerol or propylene glycol, or a monoalcohol, preferably ethanol.

### Fatty Phase

The cosmetic composition of the present invention further comprises a dispersed fatty phase.

The fatty phase is present in an amount ranging from 1% to 90% by weight, preferably from 5% to 70% by weight, more preferably from 10% to 30% by weight, relative to the total weight of the composition.

Said fatty phase preferably comprises at least one oil. The oil can be volatile or non-volatile.

The term “oil” means a water-immiscible non-aqueous compound that is liquid at room temperature (25° C.) and at atmospheric pressure (760 mmHg).

The term “non-volatile oil” means an oil that may remain on keratin materials at room temperature and atmospheric pressure for at least several hours and that especially has a vapour pressure of less than 10⁻³ mmHg (0.13 Pa). A non-volatile oil may also be defined as having an evaporation rate such that, under the conditions defined previously, the amount evaporated after 30 minutes is less than 0.07 mg/cm².

These oils may be of plant, mineral or synthetic origin.

Preferably, said oil is selected from hydrocarbonated, silicone or fluorinated oils. The term “hydrocarbon-based oil” or “hydrocarbonated oil” means an oil formed essentially from, or even constituted by, carbon and hydrogen atoms, and optionally O and N atomes, and free of Si and F heteroatoms. Such oil can contain alcohol, ester, ether, carboxylic acid, amine and/or amide groups.

The term “silicone oil” means an oil containing at least one silicon atom, especially containing Si—O groups.

The term “fluorinated oil” means an oil containing at least one fluorine atom,

Preferably, the oil is selected from hydrocarbonated oils, preferably non-volatile.

The oil can be, for example, present in an amount ranging from 0.01% to 50% by weight, preferably from 0.05% to 30% by weight, more preferably from 0.1% to 10% by weight, relative to the total weight of the composition.

### Structuring Agent(s)

The cosmetic composition of the present invention comprises structuring agents (i) and (ii).

The composition of the present invention comprises (i) at least one structuring agent with melting point of greater than or equal to 50° C., and (ii) at least one structuring agent with a melting point of less than or equal to 45° C.

Preferably, the structuring agents (i) and (ii) are selected from waxes, pasty compounds, and mixtures thereof.

The wax under consideration in the context of the present invention is generally a lipophilic compound that is solid at room temperature (25° C.), with a solid/liquid reversible change of state, having a melting point of greater than or equal to 30° C., preferably greater than or equal to 40° C., which may be up to 200° C. and in particular up to 120° C.

The term “pasty compounds” within the meaning of the present invention is understood to mean a lipophilic fatty compound with a reversible solid/liquid change in state which exhibits, in the solid state, an anisotropic crystalline arrangement and which comprises, at a temperature of 23° C., a liquid fraction and a solid fraction. In other words, the starting melting point of the pasty compound can be less than 23° C. The liquid fraction of the pasty compound, measured at 23° C., can represent from 9 to 97% by weight of the pasty compound. At 23° C., this liquid fraction preferably represents between 15 and 85% by weight, more preferably between 40 and 85% by weight of the pasty compound.

Within the meaning of the present invention, the melting point corresponds to the temperature of the most endothermic peak observed by thermal analysis (DSC) as described in Standard ISO 11357-3: 1999. The melting point of a pasty compound can be measured using a differential scanning calorimeter (DSC), for example the calorimeter sold under the name “MDSC 2920” by TA Instruments.

The measurement protocol is as follows:

A 5 mg sample of a pasty compound placed in a crucible is subjected to a first rise in temperature ranging from −20° C. to 100° C. at a heating rate of 10° C./minute, is then cooled from 100° C. to −20° C. at a cooling rate of 10° C./minute and, finally, is subjected to a second rise in temperature ranging from −20° C. to 100° C. at a heating rate of 5° C./minute. During the second rise in temperature, the variation in the difference in power absorbed by the empty crucible and by the crucible comprising the sample of pasty compound is measured as a function of the temperature. The melting point of the pasty compound is the value of the temperature corresponding to the tip of the peak of the curve representing the variation in the difference in power absorbed as a function of the temperature.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the latter to change from the solid state to the liquid state. The pasty compound is “in the solid state” when the whole of its mass is in the solid crystalline form. The pasty compound is “in the liquid state” when the whole of its mass is in the liquid form.

The enthalpy of fusion of the pasty compound is equal to the area under the curve of the thermogram obtained using a differential scanning calorimeter (DSC), such as the calorimeter sold under the name MDSC 2920 by TA Instruments, with a rise in temperature of 5 or 10° C. per minute, according to Standard ISO 11357-3:1999.

The enthalpy of fusion of the pasty compound is the amount of energy necessary to change the pasty compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compound measured at 32° C. preferably represents from 30 to 100% by weight of the pasty compound, preferably from 50 to 100% by weight of the pasty compound, more preferably from 60 to 100% by weight of the pasty compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The structuring agent (i) has a melting point greater than or equal to 50° C.

As illustrations that are suitable for the present invention, mention may be made especially of hydrocarbon-based waxes, for instance emulsifying waxes, such as solid fatty alcohols, beeswax, lanolin wax, Chinese insect waxes, rice bran wax, carnauba wax, candelilla wax, ouricury wax, esparto grass wax, berry wax, shellac wax, Japan wax and sumac wax; montan wax, orange wax and lemon wax, microcrystalline waxes, paraffins and ozokerite; polyethylene waxes, the waxes obtained by Fischer-Tropsch synthesis and waxy copolymers, and also esters thereof, fatty acids or esters obtained by catalytic hydrogenation of animal or plant oils containing linear or branched C₈-0₃₂ fatty chains, preferably C₁₆ to C₁₈ chains, silicone waxes and fluoro waxes and mixtures thereof.

More preferably, the structuring agent (i) is selected from solid fatty alcohols. By “solid fatty alcohol”, it is meant a fatty alcohol, linear or branched, saturated or unsaturated, that is solid at room temperature (25° C.) and at atmospheric pressure (780 mmHg or 1 atm.). Fatty alcohols are water-insoluble, i.e. they have a solubility in water of less than 1% by mass and preferably less than 0.5% by weight.

Preferably, the solid fatty alcohols are of structure R—OH with R denoting a saturated or unsaturated, linear alkyl group, optionally substituted with one or more hydroxyl groups, comprising from 14 to 30 carbon atoms.

Preferably, the structuring agent (i) is a solid fatty alcohol selected from solid fatty alcohols having from 14 to 30 carbon atoms.

Preferably, solid fatty alcohols having from 14 to 30 carbon atoms may be selected from myristyl alcohol (1-tetradecanol), cetyl alcohol (1-hexadecanol), palmitoleyl alcohol (cis-9-hexadecen-1-ol), stearyl alcohol (1-octadecanol), arachidyl alcohol (1-eicosanol), behenyl alcohol (1-docosanol), erucyl alcohol (cis-13-docosen-1-ol), lignoceryl alcohol (1-tetracosanol), ceryl alcohol (1-hexacosanol), myricyl alcohol and melissyl alcohol (1-triacontanol).

More preferably, according to the present invention, the solid fatty alcohol is selected from alcohols having from 14 to 22 carbon atoms, such as cetyl alcohol (1-hexadecanol), stearyl alcohol (1-octadecanol), arachidyl alcohol (1-eicosanol), behenyl alcohol (1-docosanol) and mixtures thereof.

More preferably, the solid fatty alcohol is selected from cetyl alcohol (1-hexadecanol), stearyl alcohol (1-octadecanol), arachidyl alcohol (1-eicosanol), behenyl alcohol (1-docosanol) and mixtures thereof.

When the solid fatty alcohol is a mixture, it means that several species may coexist in a commercial product, especially of different chain lengths, in the form of a mixture.

Mentions of solid fatty alcohols may be cetyl alcohol sold under the name Lanette® 16 by BASF, or behenyl alcohol sold under the name Lanette® 22 by the company BASF, or a mixture thereof.

According to one embodiment, the structuring agent (i) is present in an amount ranging from 0.01% to 10%, preferably from 0.05 to 5% by weight, more preferably from 0.1% to 3% by weight, relative to the total weight of the composition.

The structuring agent (ii) has a melting point of less than or equal to 45° C.

Preferably, the melting point of the structuring agent (ii) is between 38° C. to 45° C. Preferably, it may be a wax, a pasty compound, or a mixture thereof.

Preferably, it is a wax having a melting point of between 38° C. to 45° C., such as linear esters.

Linear esters that are suitable for use in the present invention are preferably selected from the group consisting of stearyl stearate, tetradecyl tetradecanoate (INCI name: myristyl myristate), cetyl myristate, stearyl myristate, myristyl palmitate, stearyl palmitate, myristyl stearate, cetyl stearate, stearyl stearate and cetyl palmitate, and mixtures thereof.

More particularly, tetradecyl tetradecanoate, such as the one sold under the name Tegosoft MM by the company Evonik Goldschmidt, is used in the composition of the present invention.

Preferably, the structuring agent (ii) is present in an amount ranging from 0.1% to 5%, preferably from 0.2 to 2% by weight, more preferably from 0.5% to 1.5% by weight, relative to the total weight of the composition.

### Amino Acid Surfactant

The composition of the present invention comprises at least one amino acid surfactant.

In one embodiment, said amino acid surfactant is derived from a carboxylate salt of amino acid wherein the amine group situated on the α-carbon or β-carbon of an amino acid salt is acylated with a C₈ to C₂₂ fatty acid derivative.

The carboxylate salts of these amino acids can be formed by conventional means such as by neutralization of the respective amino acid with a base. The amine group situated on the α-carbon or β-carbon of the neutralized amino acid is acylated with a fatty acid halide (acyl halide) in the presence of a base via the well-known Schotten-Baumann reaction giving the amide, thus forming the desired surfactant reaction product, i.e. the amino acid surfactant. Suitable acyl halides for acylation of the amino acid carboxylate salt include acyl chlorides, bromides, fluorides, and iodides. The acyl halides can be prepared by reacting a saturated or unsaturated, linear or branched C₈ to C₂₂ fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in U.S. Patent Application Publication No. 2008/0200704, published on Aug. 21, 2008, which application is incorporated herein by reference.

In one embodiment, said amino acid surfactant is represented by the formula (I):

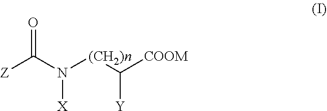

wherein:

Z represents a saturated or unsaturated, linear or branched hydrocarbon group having 8 to 22 carbon atoms,

X is hydrogen or methyl group,

n is 0 or 1,

Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)OH, —(CH₂)₂C(O)O⁻M⁺, and

M is a salt-forming cation, such as for example sodium, potassium, ammonium, or triethanolamine.

In one embodiment, in formula (I):

Z represents a linear or branched C₈ to C₂₂ alkyl or alkenyl group,

X is a hydrogen or methyl group,

n is 0,

Y is selected from hydrogen, —(CH₂)₂C(O)OH, —(CH₂)₂C(O)O⁻M⁺, and

M is a salt-forming cation, such as for example sodium, potassium, ammonium, or triethanolamine.

According to a preferred embodiment of the present invention, in the amino fatty acid of formula (I):

Z represents a linear or branched C₈ to C₂₂ alkyl or alkenyl group,

X is a hydrogen or methyl group,

n is 0,

Y is selected from hydrogen, —(CH₂)₂C(O)OH, —(CH₂)₂C(O)O⁻M⁺, and

M is a salt-forming cation, such as for example sodium, potassium, ammonium, or triethanolamine.

Examples of the amino acid surfactants are salt of alanine, arginine, aspartic acid, glutamic acid, glycine, isoleucine, leucine, lysine, phenylalanine, serine, tyrosine, valine, sarcosine, and any mixture thereof.

More specifically, mentions can be made of the amino acid surfactants such as dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

More preferably, the amino acid surfactant used in the composition is selected from salts of glutamic acid, in particular, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate.

Preferably, the amino fatty acid surfactant is present in the composition in an amount ranging from 0.1% to 5% by weight, preferably from 0.2% to 3% by weight, more preferably from 0.3% to 2% by weight, relative to the total weight of the composition.

### Non-Ionic Surfactant of Ester Type

The composition according to the present invention comprises at least one non-ionic surfactant of ester type, which is particularly a mixture of at least an ester obtained by esterification of a solid wax with a polyol, of a fatty acid diester with a polyglycerol, of a jojoba wax (preferably a jojoba wax ester), and of a fatty alcohol. Said ester is non-ionic. Said non-ionic surfactant of ester type comprises a mixture of at least one monounsaturated ester and at least one polyglyceryl diester.

According to an embodiment, the non-ionic surfactant of ester type comprises:

i) at least one monounsaturated ester of formula (II),

R1—C(O)—O—R2 (II)

wherein:

R1 and R2 represent, respectively, a C18 to C44 fatty chain, at least one of R1 or R2 is monounsaturated;

ii) at least one polyglyceryl diester of formula (III),

R3—C(O)—(O—CH2—CH(OH)—CH2)n-O—C(O)—R4 (III)

wherein:

R3 and R4 represent, respectively, a saturated C18 to C44 fatty chain, linear or branched, and

iii) at least one C10-C30 fatty alcohol.

According to an embodiment, in the formula (II), R1 and R2 represent, respectively, a C18-C40 fatty chain, more preferably a C18-C30 fatty chain. At least one of R1 or R2 is monounsaturated.

More specifically, in formula (II), the R1—C(O)- group corresponds to the carbon chain of the fatty acid. This chain may be linear or monounsaturated, and comprises at least 18 carbon atoms. Mention can be made of oleic (C18:1), gadoleic (C20:1), erucic (C22:1) acid, up to hexaconenoic (C26:1) acid for unsaturated acids. The R1—C(O) group may also consist of branched and saturated acids of at least 18 carbon atoms, also called Guerbet acids. The R2—O- group may consist of monounsaturated linear fatty alcohols with at least 18 carbon atoms. Mention can therefore be made of octadecenol, eicosenol, docosenol and hexacosenol. The carbon chain of the alcohol may also be branched and saturated and comprise at least 18 carbon atoms. Such alcohols are also called Guerbet alcohols.

Preferably, the monounsaturated ester of the formula (II) is a mixture of esters comprising various lengths of fatty chains in their structures. More preferably, such a monounsaturated ester is liquid at ambient temperature.

A preferred monounsaturated ester can be mentioned is, for example, the product commonly called jojoba oil (or jojoba esters), the liquid nature being due to the presence of monounsaturated chains. This oil comprises in particular C18:1 (preferably minority), C20:1 and C22:1 (preferably majority with C20:1>C22:1) unsaturated fatty acid esters, with C20:1, C22:1 and C24:1 unsaturated fatty alcohols.

According to an embodiment, in the formula (III), the R3—C(O)- group corresponds to the carbon chain of C18 to C44 fatty acid, said acid usually being linear and saturated, preferably corresponds to a linear and saturated C20 to C34 fatty acid. This therefore includes eicosanoic (or arachidic) acid (C20), docosanoic (or behenic) acid (C22), tetracosanoic (or lignoceric) acid (C24), hexacosanoic (or cerotic) acid (C26). The R4 group corresponds to the hydrocarbon chain of the alcohol, said alcohol usually being saturated linear and having a C18 to C44 chain, preferably C20 to C34 chain. n is an integer between 2 to 6.

According to the present invention, the polyglyceryl diester is obtained by esterification of a solid wax in the presence of at least one polyol.

Depending on the source of the wax, the mixture of monoesters may also contain a certain proportion of hydroxyacid esters such as hydroxypalmitic or hydroxystearic acid.

This is the case for example of beeswax. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes. Preferably, the solid wax is beeswax.

Solid waxes suitable for obtaining the polyglyceryl diester have a melting point between 50 and 90° C. They correspond to mixtures essentially comprising monoesters having the formula R¹—C(O)—O—R², where the R¹—C(O)- group corresponds to the carbon chain of the fatty acid, said acid usually being linear and saturated and having a number of carbon atoms of at least 18, and in particular 20, and preferably up to 44 and preferably 34. This therefore includes eicosanoic (or arachidic) acid (C20), docosanoic (or behenic) acid (C22), tetracosanoic (or lignoceric) acid (C24), hexacosanoic (or cerotic) acid (C26). Depending on the source of the wax, the mixture of monoesters may also contain a certain proportion of hydroxyacid esters such as hydroxypalmitic or hydroxystearic acid. This is the case for example of beeswax. The R² group corresponds to the hydrocarbon chain of the alcohol, said alcohol usually being saturated linear and having a number of carbon atoms of at least 18, and in particular 20, and preferably up to 44 and preferably 34. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes.

Preferably, the solid wax suitable for the esterification reaction is beeswax.

Preferably, the polyol used for esterification is selected from the group comprising ethylene glycol, diethylene glycol, triethylene glycol, 2-methyl propanediol, propylene glycol, butylene glycol, neopentyl glycol, hexylene glycol, octylene glycol, polyethylene glycol, polypropylene glycol, trimethylol propane, sorbitol, erythritol, pentaerythritol, dipentaerythritol, glycerol, diglycerol and polyglycerol (i.e. a polymer of glycerol units). More preferably, the polyol is a polyglycerol, having an average degree of polymerization between 2 and 6, preferably of 3. Preferably, the polyol is polyglycerol-3.

The non-ionic ester surfactant also comprises the acid part of a solid wax. Waxes have a complex composition. They have the common feature of containing a mixture of acid monoesters and very long chain fatty alcohols.

Preferably, the non-ionic ester surfactant is a wax derivative obtained by reacting together at least one solid wax and at least one monounsaturated ester of formula (A) in the presence of at least one polyol and optionally at least one catalyst. In such a case, a transesterification reaction occurs between the various chemical entities yielding the wax derivative.

The preferred catalysts are hydroxides or alkaline or alkaline earth alkoxides, calcium hydroxide, potassium or sodium carbonates or catalysts based on tin or titanium.

Preferably, the solid wax is advantageously selected from the group comprising carnauba wax, candelilla wax, rice bran wax, sunflower wax, sugarcane wax, ouricury wax, beeswax and Shellac wax.

In a preferred embodiment, the wax derivative is obtained by reacting jojoba oil (also called as jojoba wax), beeswax and a polyglycerol, such as polyglycerol-3.

In practice, the reaction is preferably conducted at a temperature of between 100° C. and 220° C., advantageously between 150° C. and 200° C. Preferably, the monounsaturated ester/solid wax mass ratio varies between 5/95 and 95/5, advantageously between 30/70 and 75/25. The esters of formulas (II) and (III)/polyol mass ratio preferably varies between 1/99 and 99/1, advantageously between 95/5 and 50/50. Preferably, the proportion of esterified polyol represents between 0.5 and 50% by weight of the mixture, the proportion of esterified fatty acids represents between 20 and 60% by weight of the mixture and the proportion of esterified fatty alcohols between 20 and 60% by weight of the mixture.

Preferably, the non-ionic ester surfactant is further present with a diester of a C₁₄-C₂₂ fatty acid with a polyglycerol.

Typically, the C₁₄-C₂₂ fatty acid may be selected from the group of myristic acid, stearic acid, isostearic acid, palmitic acid, oleic acid, behenic acid, erucic acid and arachidic acid, and mixtures thereof.

The polyglycerol may be a polymer of glycerol units, preferably a polymer having an average degree of polymerization between 4 and 8, preferably of 6.

Preferably, said diester is a diester of distearic acid with hexaglycerol. Preferably, it is polyglyceryl-6 distearate.

According to one particular mode of the present invention, the non-ionic ester type surfactant according to the present invention comprises at least one fatty alcohol containing from 10 to 30 carbon atoms.

As examples of fatty alcohols that may be used, mention may be made of linear or branched fatty alcohols, of synthetic origin or alternatively of natural origin, for instance alcohols originating from vegetable material (coconut, palm kernel, palm, etc.) or animal material (tallow, etc.). Use is preferably made of a fatty alcohol comprising from 20 to 26 carbon atoms, preferably from 10 to 24 carbon atoms and more preferentially from 12 to 22 carbon atoms.

As particular examples of fatty alcohols that may be used in the context of the present invention, mention may in particular be made of lauryl alcohol, myristyl alcohol, cetyl alcohol, stearyl alcohol, isostearyl alcohol, palmityl alcohol, oleyl alcohol, cetearyl alcohol (mixture of cetyl alcohol and stearyl alcohol), behenyl alcohol, erucyl alcohol and arachidyl alcohol, and mixtures thereof.

In addition, it is particularly advantageous, according to the present invention, to use together a mixture of polyglyceryl-6 distearate and polyglyceryl-3 beeswax, with cetyl alcohol and jojoba wax. Among the mixtures that are particularly preferred, mention may be made of the product sold by the company Gattefosse under the name Emulium® Mellifera, comprising jojoba wax, cetyl alcohol, polyglyceryl-6 distearate, and polyglyceryl-3 beeswax (INCI name: Polyglyceryl-6 Distearate (and) Jojoba Esters (and) Polyglyceryl-3 Beeswax (and) Cetyl Alcohol). Said mixture comprises from 5 to 30% by weight of the total weight of the mixture of jojoba wax; from 3 to 15% by weight of cetyl alcohol; at least 50% by weight of polyglyceryl-6 distearate; and from 3 to 15% by weight of polyglyceryl-3 beeswax.

The non-ionic ester surfactant may be present in a composition of the present invention in an amount ranging from 0.1% to 10% by weight, preferably from 0.5% to 5% by weight, relative to the total weight of the composition.

### Surfactant of Alkylpolyglycoside Type

According to one embodiment, the composition according to the present invention further comprises at least one surfactant of alkylpolyglycoside type.

For the purposes of the present invention, the term “alkylpolyglycoside” is intended to mean an alkylmonosaccharide (degree of polymerization is 1) or an alkylpolysaccharide (degree of polymerization greater than 1).

The alkylpolyglycosides may be used alone or in the form of mixtures of several alkylpolyglycosides. They generally correspond to formula (IV):

R(O)(G)ₓ (IV)

in which the radical R is a linear or branched C₁₂^{-C}₂₂ alkyl radical, preferably a C₁₂-C₂₀ alkyl radical, G is a saccharide residue and x ranges from 1 to 5, preferably from 1.05 to 2.5 and more preferentially from 1.1 to 2.

The saccharide residue may be selected from the group of glucose, dextrose, saccharose, fructose, galactose, maltose, maltotriose, lactose, cellobiose, mannose, ribose, dextran, talose, allose, xylose, levoglucan, cellulose and starch. More preferentially, the saccharide residue denotes glucose.

It should also be noted that each unit of the polysaccharide part of the alkylpolyglycoside may be in α or β isomer form, in L or D form, and the configuration of the saccharide residue may be of furanoside or pyranoside type.

It is, of course, possible to use mixtures of alkylpolysaccharides, which may differ from each other in the nature of the borne alkyl unit and/or the nature of the bearing polysaccharide chain.

In addition, it is particularly advantageous, according to the present invention, to use together a fatty alcohol and an alkylpolyglycoside of which the alkyl part is identical to that of the selected fatty alcohol.

Fatty alcohol/alkylpolyglycoside emulsifying mixtures as defined are described in particular in patent applications WO 92/06778, WO 95/13863 and WO 98/47610.

Among the fatty alcohol/alkylpolyglycoside mixtures that are particularly preferred, mention may be made of the products sold by the company SEPPIC under the name Montanov®, such as the following mixtures:
cetylstearyl alcohol/cocoyl glucoside—Montanov 82®,
arachidyl alcohol and behenyl alcohol/arachidyl glucoside—Montanov 802®,
myristyl alcohol/myristyl glucoside—Montanov 14®,
cetylstearyl alcohol/cetylstearyl glucoside—Montanov 68®,
C₁₄^{-C}₂₂ alcohol/C₁₂-C₂₀ alkyl glucoside—Montanov L®,
cocoyl alcohol/cocoyl glucoside—Montanov S®, and
isostearyl alcohol/isostearyl glucoside—Montanov WO 18®.

According to one particular embodiment, the alkylpolyglycoside used in a composition according to the present invention is C₁₂-C₂₀ alkylglucoside. It is advantageously used as a mixture with a C₁₄-C₂₂ alcohol.

According to one particular embodiment of the present invention, use is thus made of the C₁₄-C₂₂ alcohol/C₁₂-C₂₀ alkylglucoside mixture, such as the product sold by the company SEPPIC under the name Montanov 68®, consisting of approximately 20% of C₁₂-C₂₀ alkylglucoside and of approximately 80% of C₁₄-0₂₂ alcohol.

If present, the surfactant of alkylpolyglycoside type is present in an amount ranging from 0.1% to 3%, preferably from 0.1 to 1% by weight, more preferably from 0.1% to 0.5% by weight, relative to the total weight of the composition

### Acidic Cosmetic Active Ingredients

The cosmetic composition of the present invention comprises at least one acidic cosmetic active ingredient.

The term “acidic cosmetic active ingredient” means any cosmetic active ingredient the dispersion or solution thereof in a solvent has an acidic pH.

As examples of acidic cosmetic active ingredient, mention can be made of saccharomyces/xylinum/black tea ferment, vaccinium myrtillus fruit extract, saccharum officinarum (sugarcane) extract, citrus aurantium dulcis (orange) fruit extract, citrus limon (lemon) fruit extract, acer saccharum (sugar maple) extract, glycolic acid, hydroxypropyl tetrahydropyrantriol, etc.

According to one embodiment, the acidic cosmetic active ingredient is present in an amount ranging from 0.1% to 15%, preferably from 1 to 12% by weight, more preferably from 3% to 10% by weight, relative to the total weight of the composition.

### Additional Ingredients

The cosmetic composition of the present invention may comprise at least one additive.

A person skilled in the art can adjust the type and amount of additives present in the compositions according to the present invention by means of routine operations, so that the desired cosmetic properties and stability properties for these compositions are not affected by the additives. Said additive may be selected from thickeners, active agents and preservatives.

According to a particularly preferred embodiment, the present invention relates to a cosmetic composition in form of oil-in-water emulsion, comprising, relative to the total weight of the composition:

(i) from 0.1% to 3% by weight of at least one structuring agent selected from a solid fatty alcohol having from 14 to 30 carbon atoms, preferably selected from myristyl alcohol, cetyl alcohol, palmitoleyl alcohol, stearyl alcohol, arachidyl alcohol, behenyl alcohol, erucyl alcohol, lignoceryl alcohol, ceryl alcohol, myricyl alcohol and melissyl alcohol,

(ii) from 0.5% to 1.5% by weight of at least one structuring agent selected from stearyl stearate, tetradecyl tetradecanoate, cetyl myristate, stearyl myristate, myristyl palmitate, stearyl palmitate, myristyl stearate, cetyl stearate, stearyl stearate and cetyl palmitate,

(iii) from 0.3% to 2% by weight of at least one amino acid surfactant selected from salts of glutamic acid, in particular, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate;

(iv) from 0.5% to 5% by weight of at least one non-ionic surfactant of ester type, comprising a mixture of polyglyceryl-6 distearate, jojoba esters, polyglyceryl-3 beeswax, and cetyl alcohol;

(v) from 3% to 10% by weight of at least one acidic cosmetic active ingredient selected from saccharomyces/xylinum/black tea ferment, vaccinium myrtillus fruit extract, saccharum officinarum (sugarcane) extract, citrus aurantium dulcis (orange) fruit extract, citrus limon (lemon) fruit extract, acer saccharum (sugar maple) extract, glycolic acid, and hydroxypropyl tetrahydropyrantriol; and

(vi) optional, from 0.1% to 0.5% by weight of at least one surfactant of alkylpolyglycoside type selected from C₁₂-C₂₀ alkylglucoside.

### Method and Use

According to an embodiment, the present invention relates to a non-therapeutic method for treating a keratin material, comprising the step of applying the composition of the present invention to the keratin material.

Preferably the present invention relates to a method for caring for the skin, comprising the step of applying the composition of the present invention to the skin.

The following examples serve to illustrate the present invention without, however, being limiting in nature.

### EXAMPLES

### Example 1

### Preparation of Cosmetic Compositions

Cosmetic compositions according to comparative formula (comp.) and inventive formulas (invt.) were prepared according to the amounts given in the table below. The amounts are given in % by weight of the total composition.

| Name | Commercial Name | Supplier | Comp. | Invt. 1 | Invt. 2 |
|---|---|---|---|---|---|
| DISODIUM EDTA | DISSOLVINENA-2 | AKZO NOBEL | 0.1 | 0.1 | 0.1 |
| BETAINE | AMINOCOAT | ASAHI KASEI | 2 | 2 | 2 |
| CETYL ALCOHOL | LANETTE ® 16 | BASF | 0.5 | 0.5 | 0.75 |
| DI-C12-13 ALKYL MALATE | COSMACOL EMI | SASOL | 2 | 2 | 2 |
| DIISOPROPYL SEBACATE | DUB DIS | STEARIN ERIE | 2 | 2 | 3 |
| | | DUBOIS | | | |
| MYRISTYL MYRISTATE | TEGOSOFT MM | EVONIK | 1 | 1 | 1 |
| | | GOLDSCHMIDT | | | |
| BEHENYL ALCOHOL | LAN ETTE ® 22 | BASF | 0.5 | 0.5 | 0.5 |
| CETEARYL ALCOHOL (and) | MONTANOV ™ 68 | SEPPIC | 0.3 | 0.3 | |
| CETEARYL GLUCOSIDE | | | | | |
| DISODIUM STEAROYL | | | | 0.5 | 0.5 |
| GLUTAMATE | | | | | |
| POLYGLYCERYL-6 | EMULIUM ® | GATTEFOSSE | 3 | 3 | 3 |
| DISTEARATE (and) JOJOBA | MELLIFERA | | | | |
| ESTERS (and) CETYL | | | | | |
| ALCOHOL (and) | | | | | |
| POLYGLYCERYL-3 | | | | | |
| BEESWAX | | | | | |
| GLYCERIN | GLYCERINE CP | VVF | 7 | 7 | 6.8 |
| PROPANEDIOL | ZEMEA | DUPONT TATE | 3 | 3 | 3 |
| | PROPANEDIOL | AND LYLE BIO | | | |
| | | PRODUCTS | | | |
| PHENOXYETHANOL | | | 0.4 | 0.39 | 0.39 |
| CHLORPHENESIN | | | | 0.2 | 0.2 |
| SACCHAROMYCES/XYLINU | KOMBUCHKA | SEDERMA | 3.5 | 3.5 | 3.5 |
| M/BLACK TEA FERMENT | | (CRODA) | | | |
| (and) GLYCERIN | | | | | |
| VACCINIUM MYRTILLUS | ACB FRUIT MIX | ACTIVE | 0.01 | 0.01 | |
| FRUIT EXTRACT (and) OR | CONCEPTS | LLC | | | |
| SACCHARUM | | | | | |
| OFFICINARUM | | | | | |
| (SUGARCANE) EXTRACT | | | | | |
| (and) CITRUS AURANTIUM | | | | | |
| DULCIS (ORANGE) FRUIT | | | | | |
| EXTRACT (and) CITRUS | | | | | |
| LIMON (LEMON) FRUIT | | | | | |
| EXTRACT (and) ACER | | | | | |
| SACCHARUM (SUGAR | | | | | |
| MAPLE) EXTRACT | | | | | |
| GLYCOLIC ACID | GLYPURE 70 | DUPONT | 0.01 | 0.01 | |
| HYDROXYPROPYL | MEXORYL SCN | CHIMEX | 3.15 | 3.15 | 1.05 |
| TETRAHYDROPYRANTRIOL | | | | | |
| WATER | | | QS to | QS to | QS to |
| | | | 100 | 100 | 100 |

### Example 2

### Evaluation

The stability, usage and cosmetic properties of cosmetic compositions prepared in Example 1 were measured.

The stability tests of the cosmetic compositions according to invention and comparative formulas at 40° C., 45° C., and 65° C. for two months were conducted using Binder oven (USA), by leaving the invention and comparative formulas in the oven for 2 months.

The stability tests at 4° C. stability for two months were conducted using Zhongke Meiling refrigerator (YC-260L, China), by leaving the cosmetic compositions according to invention and comparative formulas in the refrigerator for 2 months.

The light stability tests for 24 hours were conducted using ATLAC (AMETEK Measurement and Calibration Technologies).

Lastly, the freezing-thaw stability tests were conducted for 10 cycles using Binder over (USA). In each cycle, the temperature will be changed gradually from 20° C. to −20° C. in 24 hours.

Then the cosmetic compositions of inventive and comparative formulas were given to 5 consumers to be applied on the skin, and score of easy spreading, as well as fresh and moisturizing feeling were given by the consumers:

1: very poor;

2: little effect, not acceptable;

3: acceptable;

4: good effect;

5: very good effect, very easy to pick up and spread, fresh on the skin.

The result of the stability, usage and cosmetic properties of the compositions according to comparative formula and inventive formulas were listed below.

| Test | | Comp. | Invt. 1 | Invt. 2 |
|---|---|---|---|---|
| Stability test | 4° C. | Pass | Pass | Pass |
| at different | 40° C. | Not stable* | Pass | Pass |
| temperatures | 45° C. | Not stable* | Pass | Pass |
| in 2 months | 65° C. | Not stable* | Pass | Pass |

| | | | |
|---|---|---|---|
| Light stability test | Pass | Pass | Pass |
| Freezing-shaw stability test | Pass | Pass | Pass |
| Easy spreading | 5 | 5 | 5 |
| Fresh and moisturizing feeling | 4 | 4 | 4 |

The appearance of the cosmetic composition according to the comparative formula is not good upon formulation, detailed status could be observed through microscope. Moreover, the cosmetic composition according to the comparative formula got worse apparently at high temperature less than 2 months.

It was observed with microscopy that the oil droplets are not dispersed evenly in the cosmetic composition according to comparative formula, and the size of them are over 10 μm partially, impairing the stability, as shown in FIG. 1. It was also observed that the droplets in the cosmetic compositions according to inventive formulas 1 and 2 are much more evenly dispersed and the particle size of them is much smaller, as shown in FIGS. 2 and 3.

The cosmetic compositions of inventive formulas 1 and 2 are easy to spread on the skin, and provides to the skin fresh and moisturizing feeling. In addition, the cosmetic compositions of inventive formulas 1 and 2 are stable.

1. A cosmetic composition in form of oil-in-water emulsion, comprising:
(i) at least one structuring agent with a melting point of greater than or equal to 50° C.,
(ii) at least one structuring agent with a melting point of less than or equal to 45° C.,
(iii) at least one amino acid surfactant;
(iv) at least one non-ionic surfactant of ester type, comprising a mixture of at least one monounsaturated ester and at least one polyglyceryl diester; and
(v) at least one acidic cosmetic active ingredient.

2. The cosmetic composition of claim 1, wherein the structuring agent (i) is selected from the group consisting of hydrocarbon-based waxes, montan wax, orange wax and lemon wax, microcrystalline waxes, paraffins and ozokerite; polyethylene waxes, the waxes obtained by Fischer-Tropsch synthesis and waxy copolymers, and also esters thereof, fatty acids or esters obtained by catalytic hydrogenation of animal or plant oils containing linear or branched C₈-C₃₂ fatty chains, silicone waxes, fluoro waxes and solid fatty alcohols of structure R—OH, with R denoting a saturated or unsaturated, linear alkyl group, optionally substituted with one or more hydroxyl groups, comprising from 14 to 30 carbon atoms.

3. The cosmetic composition of claim 1, wherein the structuring agent (i) is present in an amount ranging from 0.01% to 10% by weight, relative to the total weight of the composition.

4. The cosmetic composition of claims 1, wherein the structuring agent (ii) is selected from the group consisting of stearyl stearate, tetradecyl tetradecanoate, cetyl myristate, stearyl myristate, myristyl palmitate, stearyl palmitate, myristyl stearate, cetyl stearate, stearyl stearate and cetyl palmitate.

5. The cosmetic composition of claims 1, wherein the structuring agent (ii) is present in an amount ranging from 0.1% to 5%, relative to the total weight of the composition.

6. The cosmetic composition of claims 1, wherein the amino acid surfactant is chosen from compound of formula (I), 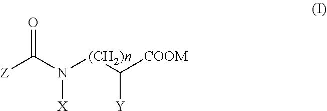
wherein in formula (I):
Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M′, —(CH₂)₂C(O)OH, —(CH₂)₂C(O)O⁻M′, and
M is a salt-forming cation.

7. The cosmetic composition as claimed in claim 6, wherein the amino acid surfactant is selected from salt of alanine, arginine, aspartic acid, glutamic acid, glycine, isoleucine, leucine, lysine, phenylalanine, serine, tyrosine, valine, sarcosine, and any mixture thereof.

8. The cosmetic composition as claimed in claims 1, wherein the amino acid surfactant is present in an amount ranging from 0.1% to 5% by weight relative to the total weight of the composition.

9. The cosmetic composition as claimed in claim 1, wherein said non-ionic surfactant of ester type comprises:
i) at least one monounsaturated ester of formula (II),
R1—C(O)—O—R2 (II)
wherein:
R1 and R2 represent, respectively, a C18 to C44 fatty chain, at least one of R1 or R2 is monounsaturated;
ii) at least one polyglyceryl diester of formula (III),
R3—C(O)-(O—CH2—CH(OH)-CH2)n-O—C(O)—R4 (III)
wherein:
R3 and R4 represent, respectively, a saturated C18 to C44 fatty chain, linear or branched, and
iii) at least one C10-C30 fatty alcohol.

10. The cosmetic composition of claim 1, wherein the non-ionic surfactant of ester type is a mixture of polyglyceryl-6 distearate, jojoba esters, polyglyceryl-3 beeswax, and cetyl alcohol.

11. The cosmetic composition of claims 1, wherein the non-ionic ester surfactant is present in an amount ranging from 0.1% to 10% by weight relative to the total weight of the composition.

12. The cosmetic composition of claim 1, wherein the acidic cosmetic active ingredient is selected from the group consisting of saccharomyces/xylinum/black tea ferment, vaccinium myrtillus fruit extract, saccharum officinarum (sugarcane) extract, citrus aurantium dulcis (orange) fruit extract, citrus limon (lemon) fruit extract, acer saccharum (sugar maple) extract, glycolic acid and hydroxypropyl tetrahydropyrantriol.

13. The cosmetic composition of claim 1, wherein the acidic cosmetic active ingredient is present in an amount ranging from 0.1% to 15% relative to the total weight of the composition.

14. The cosmetic composition as claimed in claim 13, wherein it further comprises at least one surfactant of alkylpolyglycoside type selected from the compound of formula (IV):
R(O)(G)ₓ (IV)
in which the radical R is a linear or branched C₁₂-C₂₂ alkyl radical, x ranges from 1 to 5 and G is a saccharide residue selected from the group consisting of glucose, dextrose, saccharose, fructose, galactose, maltose, maltotriose, lactose, cellobiose, mannose, ribose, dextran, talose, allose, xylose, levoglucan, cellulose and starch.

15. Cosmetic composition in form of oil-in-water emulsion, comprising, relative to the total weight of the composition:
(i) from 0.1% to 3% by weight of at least one structuring agent selected from a solid fatty alcohol having from 14 to 30 carbon atoms,
(ii) from 0.1% to 1.5% by weight of at least one structuring agent selected from stearyl stearate, tetradecyl tetradecanoate, cetyl myristate, stearyl myristate, myristyl palmitate, stearyl palmitate, myristyl stearate, cetyl stearate, stearyl stearate and cetyl palmitate,
(iii) from 0.3% to 2% by weight of at least one amino acid surfactant selected from salts of glutamic acid;
(iv) from 0.5% to 5% by weight of at least one non-ionic surfactant of ester type, comprising a mixture of polyglyceryl-6 distearate, jojoba esters, polyglyceryl-3 beeswax, and cetyl alcohol;
(v) from 3% to 10% by weight of at least one acidic cosmetic active ingredient selected from the group consisting of saccharomyces/xylinum/black tea ferment, vaccinium myrtillus fruit extract, saccharum officinarum (sugarcane) extract, citrus aurantium dulcis (orange) fruit extract, citrus limon (lemon) fruit extract, acer saccharum (sugar maple) extract, glycolic acid, and hydroxypropyl tetrahydropyrantriol; and
(vi) optionally, from 0.1% to 0.5% by weight of at least one surfactant of alkylpolyglycoside type selected from C₁₂-C₂₀ alkylglucoside.

16. A method for caring for the skin, comprising the application to the skin of the cosmetic composition of claim 1.
